(19) United States
(12) Patent Application Publication
(10) Pub. No.: US 2013/0110085 A1
(43) Pub. Date: May 2, 2013

(54) Catheter With Proximally Adjustable Length

(71) Applicant: Josephine Branch Craige Adamson, Virginia Beach, VA (US)

(72) Inventor: Josephine Branch Craige Adamson, Virginia Beach, VA (US)

(21) Appl. No.: 13/281,431

(22) Filed: Oct. 26, 2011

(51) Int. Cl. A61M 25/00

(52) U.S. Cl. 604/523

(57) ABSTRACT

A catheter and associated method. A catheter designed for accessing a tissue location in the human body, typically a location within the vascular system or accessible through the vasculature. The catheter has an adjustable region(s) between its hub or proximal terminating unit and the surface of the patient's body which enables the physician to elongate or shorten the catheter through direct manipulation, either during catheter placement or after the catheter has been deployed within the body. The adjustable region(s) are lined with an elastic membrane that maintains a uniform inner diameter as the catheter length changes. The catheter maintains a continuous smooth uninterrupted lumen available for the passage of other catheters and/or fluids. The catheter and method of proximal length adjustment may be applied to any catheter, including indwelling catheters, guiding catheters and microcatheters.

### CROSS-REFERENCE TO RELATED APPLICATIONS

This application claims priority as a utility application of U.S. 61/455,609 and U.S. 61/455,610, both filed on Oct. 25, 2010, the contents of both of which are incorporated herein in their entirety by reference.

### TECHNICAL FIELD OF THE INVENTION

The technical field of the invention relates to configurations and methods for extending the length of a catheter, especially providing the ability to extend the length of the catheter without need to remove the catheter from the patient during a procedure. The technical field of the invention also relates to a catheter designed for accessing a tissue location within the human body, typically though not limited to, a location within the vascular system or accessible through the vasculature. Specifically the invention relates to an improved catheter with a proximally adjustable length. The invention may be applied to any catheter, including indwelling catheters, guiding catheters, and microcatheters, and is not limited to a catheter used in the vascular system.

### BACKGROUND OF THE INVENTION

The number of endovascular procedures has increased dramatically in the past decade. Catheters are used within blood vessels in most organs of the body, including the heart, extremities, kidneys, and brain. These procedures have been shown to reduce the need for general anesthesia, to reduce the length of recovery time and hospital stay, and provide comparable or improved treatment outcomes. Current practice involves accessing the patients' arterial system (typically via the femoral artery) with a flexible guiding catheter, snaking the guiding catheter to the target location, and then inserting a smaller microcatheter, balloon, or other device within the guiding catheter to perform the therapeutic treatment.

By way of example, current techniques in neurointerventional surgery employ catheters of many different sizes and lengths. For many years, the standard approach consisted of placing a guiding catheter into the cervical internal carotid artery or into the cervical vertebral artery, and then placing a microcatheter or other device through this catheter and into the cerebral circulation. Typical guiding catheters were 90-100 cm in length, and microcatheters were 120-155 cm in length. Improvements in technology have made it possible to place large catheters and guiding catheters into the intracranial vessels, and guiding catheters and other larger catheters now come in various lengths from 90 cm to 135 cm. Given the relatively recent profusion of different lengths of catheters, mismatches in lengths can now unexpectedly occur such that any catheter, either a guiding catheter or a microcatheter, can be too long or too short to perform the intended procedure. A typical problem in angiographic procedures stems from the physician being forced to anticipate a catheter's needed length at the beginning of the case. If the physician judges incorrectly, and the guiding catheter or the microcatheter is too short, then the entire catheter system must be withdrawn. Typically, the physician will re-access the circulation with a different catheter, wasting time and increasing the danger of the procedure. If the catheter could be elongated at the proximal end, then it could be used without the need to remove it and place another catheter.

On the other hand, catheters occasionally prove to be too long for the intended procedure. For example if an operator chooses a 115 cm guiding catheter with the intention of placing it intracranially, and then is unable to do so, the operator is left with a catheter in the cervical carotid artery or vertebral artery. With the 115 cm guiding catheter in place proximally in the arterial system, standard length microcatheters or balloons or stents may be too short to reach distally into the circulation; the operator may not be able to reach target blood vessels that are proximal to the cervical carotid artery or the vertebral artery with a shorter microcatheter or balloon/stent. In these circumstances it would be advantageous to be able to shorten the guiding catheter at the proximal/physician's end of the catheter so that the microcatheter or balloon/stent would reach the target.

In addition, the same or similar technology can be used to proximally elongate or shorten the microcatheter/balloon catheter/stent, so that the device can reach the target even if the guiding catheter is not the proper length. The current invention solves this problem with catheters that can be proximally elongated or shortened as needed to mitigate the problem of mismatch and reduce the need to exchange guiding catheters or microcatheters/balloons/stents.

The invention can be used in the fields of cardiology, nephrology, urology, gynecology, or any field of medicine that uses catheters to access tissue targets within the human body. Eliminating the need for catheter exchange in any part of the vascular system would decrease the risks of patient blood vessel disruption and possible morbidity, (infection, scarring, plaque formation), as well as decrease the number of blood vessel dissections, air embolisms or thromboembolisms, well-known complications of changing or exchanging catheters. This proximally adjustable catheter and associated method would not only improve patient outcome, it would create cost savings: because proximal catheter adjustment increases physician efficiency, the method would decrease patient anesthesia time and provide a cost savings in OR time, anesthesia time, and discarded catheters.

### SUMMARY OF THE INVENTION

The main component of the catheter with proximally adjustable length includes a hollow flexible catheter comprised of layers of plastic, metal, or composite material with one or multiple adjustable zone(s) intermediate its ends and proximal to the operator's end of the catheter. The adjustable region is adjacent to or an integral part of the catheter hub or proximal terminating unit. The adjustable region(s) are lined with a compliant hydrophilic polymer tube. Before, during, and after length adjustment the catheter maintains a continuously patent lumen; therefore length adjustment can occur even when other devices are contained within the catheter lumen.

In one embodiment the adjustable zone is formed by a plurality of telescoping sections adapted for retraction one into the other.

In another embodiment the adjustable zone is formed by a plurality of circumferential corrugations in accordion-like ridges and grooves. The catheter may be lengthened by increasing the distance between the ridges. The catheter may be shortened by decreasing the distance between the ridges. Every ridge is comprised of a long side and a short side so that each ridge stacks neatly into the adjacent ridge. The stacked ridges provide the operator an easy grasp on the adjustable zone and the ability to change or maintain the selected catheter length in increments.

Additional embodiments could include proximal adjustable zones created with collapsible spirals or folding plates.

This catheter with adjustable length addresses the technical problem of exchanging catheters and provide a method of adjusting a catheter's length at the proximal end of the catheter as it terminates in the physician's hands. The physician is able to lengthen or shorten the catheter by direct manipulation thus extending or retracting the catheter shaft as desired. The length adjustment can occur either before, during, or after catheter deployment into the patient's body. During length adjustment the catheter lumen remains continuously patent.

In one general aspect, the invention relates to a hollow catheter having an inner lumen with an inner surface and comprising a distal catheter shaft, an adjustable length catheter portion, and a proximal hub. The adjustable length catheter portion is positioned between the distal catheter shaft and the proximal hub and includes a segment that is either expandable to extend the length of the catheter or is compressible to shorten the length of the catheter. The adjustable length catheter portion has a proximal end within about 10 cm of the hub. The adjustable length catheter portion has a continuously smooth inner surface when in the expanded or shortened configuration, the smooth inner surface being formed of a biocompatible material that is stretched when the adjustable length catheter portion is expanded and is relaxed when the adjustable length catheter portion is shortened.

Embodiments of the invention may include one or more of the following features. For example, the adjustable length catheter portion may have the proximal end within about 7 cm of the hub, within about 5 cm of the hub, within about 3 cm of the hub or within about 1 cm of the hub.

The biocompatible material forming the smooth inner surface of the adjustable length catheter portion may be a urethane, a polyether block amide, or a rubber.

The adjustable length catheter portion may have an outer catheter wall and the smooth inner surface adjacent to and coplanar with the outer catheter wall, and the outer catheter wall comprises one or more corrugations.

The adjustable length catheter portion may have a first catheter segment having a first outer diameter and a second catheter segment having an inner diameter that is greater than the first outer diameter such that the second catheter segment can telescope over the first catheter segment. The smooth inner surface is attached to the first catheter segment and the second catheter segment such that a continuous and smooth inner liner is formed when the two catheter segments are expanded and shortened.

The adjustable length catheter portion may have an adjustable zone formed by a plurality of circumferential corrugations in accordion-like ridges and grooves whereby the catheter may be lengthened by increasing the distance between the ridges or shortened by decreasing the distance between the ridges.

The ridge may be comprised of a long side and a short side such that each ridge stacks neatly into an adjacent ridge, whereby the stacked ridges provide the operator with an an easy grasp on the adjustable zone and the ability to change or maintain the selected catheter length in increments.

In another general aspect there is provided a method of adjusting a length of a catheter while in a patient, the method comprising:

providing a catheter having an inner lumen with an inner surface and including a distal catheter shaft, an adjustable length catheter portion, and a proximal hub, wherein the adjustable length catheter portion is positioned between the distal catheter shaft and the proximal hub and includes a segment that is either expandable to extend the length of the catheter or is compressible to shorten the length of the catheter, wherein the adjustable length catheter portion has a proximal end within about 10 cm of the hub, and wherein the adjustable length catheter portion has a continuously smooth inner surface when in the expanded or shortened configuration, the smooth inner surface being formed of a biocompatible material that is stretched when the adjustable length catheter portion is expanded and is relaxed when the adjustable length catheter portion is shortened;

inserting the catheter into a tubular vessel of a patient; and

adjusting the length of the catheter without removing the catheter from the vessel by adjusting the adjustable length catheter portion.

Embodiments of the invention may include one or more of the following features. For example, the adjustable length catheter portion may have the proximal end within about 7 cm of the hub, within about 5 cm of the hub, within about 3 cm of the hub or within about 1 cm of the hub.

The biocompatible material forming the smooth inner surface of the adjustable length catheter portion may be a urethane, a polyether block amide, or a rubber.

The adjustable length catheter portion may have an outer catheter wall and the smooth inner surface adjacent to and coplanar with the outer catheter wall, and the outer catheter wall comprises one or more corrugations.

The adjustable length catheter portion may have a first catheter segment having a first outer diameter and a second catheter segment having an inner diameter that is greater than the first outer diameter such that the second catheter segment can telescope over the first catheter segment. The smooth inner surface is attached to the first catheter segment and the second catheter segment such that a continuous and smooth inner liner is formed when the two catheter segments are expanded and shortened.

The adjustable length catheter portion may have an adjustable zone formed by a plurality of circumferential corrugations in accordion-like ridges and grooves whereby the catheter may be lengthened by increasing the distance between the ridges or shortened by decreasing the distance between the ridges.

The ridge may be comprised of a long side and a short side such that each ridge stacks neatly into an adjacent ridge, whereby the stacked ridges provide the operator with an an easy grasp on the adjustable zone and the ability to change or maintain the selected catheter length in increments.

The details of various embodiments of the invention are set forth in the accompanying drawings and the description below. Other features and advantages of the invention will be apparent from the description, the drawings, and the claims.

### DESCRIPTION OF THE DRAWINGS

FIG. 1 represents a lateral view of the invention demonstrating the proximal location of the adjustable zone.

FIG. 2 represents one embodiment of the adjustable zone of the invention, where said zone includes one or more sections adapted for telescoping receipt of the extendable sections. These sections can be either slidable or threaded.

FIG. 3 represents an additional embodiment of the adjustable zone, where said zone is formed by one or more regions of multiple corrugations that allow for accordion-like distraction or retraction.

FIG. 4 represents a longitudinal section of the adjustable zone demonstrating the hydrophilic compliant polymer inner membrane tube that is continuous with the inner wall of the catheter.

FIG. 5 is a cross-section side view of an adjustable length catheter portion using telescoping catheter walls with the adjustable length portion being shown in a shortened configuration.

FIG. 6 is a cross-sectional side view of the adjustable length catheter portion of FIG. 5 shown in an extended configuration.

FIGS. 7 and 8 are perspective views of clips for maintaining the length of the adjustable length portion when in an extended position.

FIG. 9 is a side view of a corrugated adjustable length catheter portion with the corrugation in a compressed configuration.

FIG. 10 is a cross-sectional side view of the corrugated adjustable length catheter portion of FIG. 9.

FIG. 11 is a side view of the corrugated adjustable length catheter portion of FIG. 9 in an extended position with hidden lines showing the position of the inner continuous, smooth lumen.

### DETAILED DESCRIPTION OF THE INVENTION

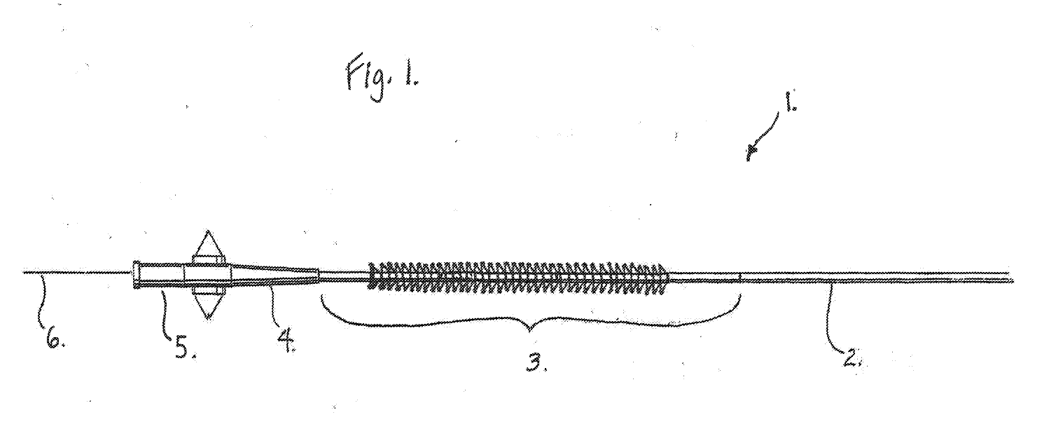

For FIGS. 1-4, catheter shaft 2 is a hollow tube comprised of layers of plastic, metal, or composite material. For example, the catheter can have an inner layer of plastic covered with a metal or polymer (e.g., Kevlar) braiding, and finally covered with an outer layer of plastic. Suitable plastics for making the catheter include nylon, urethane, polyethylene, polypropylene and the like, including mixtures of such polymers. One suitable polymer for the catheter is marketed under the name Pebax and is available in a variety of durometers. A guiding catheter may additionally have an inner layer or coating of a lubricious material, such as Teflon, i.e., polytetrafluoroethylene (PTFE).

FIG. 1 represents the proximal portion of a catheter 1. FIG. 1 demonstrates the location of the adjustable zone 3 being adjacent to or an integral part of the catheter hub 5 and strain relief 4 which together comprise the proximal terminus of the catheter as it lies in the operator's hand. Zone 3 may contain one or more adjustable units. In this representation there is a single unit of the corrugated embodiment. The guidewire 6 is shown inserted into the catheter in this embodiment of the invention as a guiding catheter. The catheter and method of proximal length adjustment may be applied to any catheter, including indwelling catheters, guiding catheters and microcatheters.

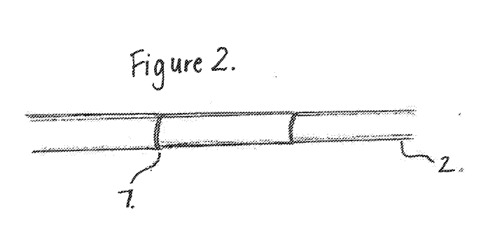

FIG. 2 demonstrates one embodiment of the adjustable zone of the catheter in detail where one or more sections 7 of the catheter are adapted for telescoping receipt of the extendable sections. These sections 7 may be slidable or threaded to facilitate speed and precision of the length adjustment.

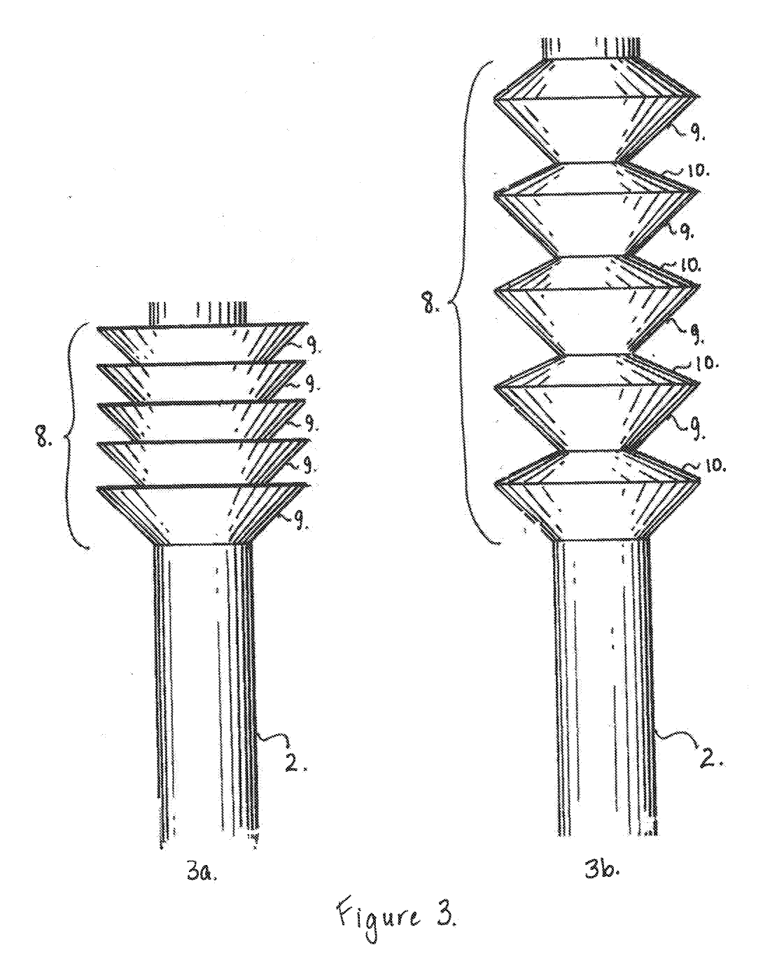

FIG. 3 represents another embodiment of the invention in detail where one or more corrugated or folded sections 8 of the catheter are formed with a plurality of circumferential corrugations (e.g., folds) in accordion-like ridges and grooves. Each corrugation or fold is comprised of a long side 9 and a short side 10. The catheter may be shortened 3a by decreasing the distance between the ridges. When fully shortened each ridge stacks neatly into the adjacent ridge. The catheter may be lengthened 3b by increasing the distance between the ridges.

FIG. 4 represents the inner membrane 11 that lines the corrugated adjustable zone 8. The membrane is comprised of a compliant hydrophilic polymer membrane. The inner membrane maintains a smooth inner lumen, contiguous with a Teflon (PTFE) inner layer or coating of the catheter shaft 2, as the corrugated adjustable zone 8 elongates or shortens.

The adjustable zone of the catheter may be separately formed and fused to a catheter shaft 2 or may be formed integrally with the catheter shaft. FIG. 5 illustrates one embodiment of the adjustable zone in the form of a proximal positioned, adjustable length catheter portion 20. The portion 20 may be integral with the catheter shaft 2 and the hub/strain relief attached to the distal end 22 of the portion 20. Alternatively, the portion 20 may be separately formed and fused to the catheter shaft using techniques well known in the catheter art, e.g., flaring one end of the first piece, forming a mating end of the second piece, inserting and applying heat to fuse the first piece to the second piece.

The portion 20 includes a first catheter segment 24 having an outer diameter and a second catheter segment 26 having an inner diameter. The inner diameter of the second segment is greater than the outer diameter of the first segment such that the second segment can telescope over the first segment. FIG. 5 also illustrates an elastic, inner liner 28 attached to the first and second catheter segments at attachment points 30. The liner may be any elastic, biocompatible material that can be stretched from a shortened position to a stretched or expanded position. Suitable polymers include polyether block amides, urethanes, silicones, latex, rubber, etc.

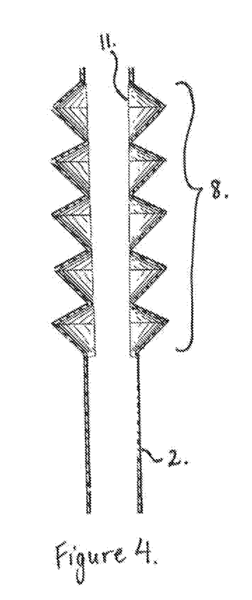

The liner is fused or otherwise attached to the inner lumen of the first and second catheter segments such that in the shortened position the liner is generally smooth. This is desired to reduce the likelihood that a thrombus will form on the surface, as could be expected if the surface had numerous folds or was too irregular. As illustrated in FIG. 6, the liner also is smooth when the first and second catheter segments are in the extended or expanded position. The liner will also be contiguous with a Teflon (PTFE) layer in the catheter shaft of the guiding catheter.

Depending upon the elasticity of the liner there may be more or less tendency for the extended catheter segments to attempt to return to a shortened configuration. To the extent such a tendency occurs and is an inconvenience to the operator, the first catheter segment may be provided with a ridge or multiple protrusions 32. The position of the ridge or protrusions will create a length between the ridge and the end of the second catheter segment into which a clip or spacer may be placed. As illustrated in FIGS. 6 and 7, a tube-like spacer 35 is positioned between the ridge 32 and the second catheter segment 26, thereby ensuring that the first and second catheter segments are maintained in an extended position. Therefore, it should be understood that the use of the clips/spacers is optional.

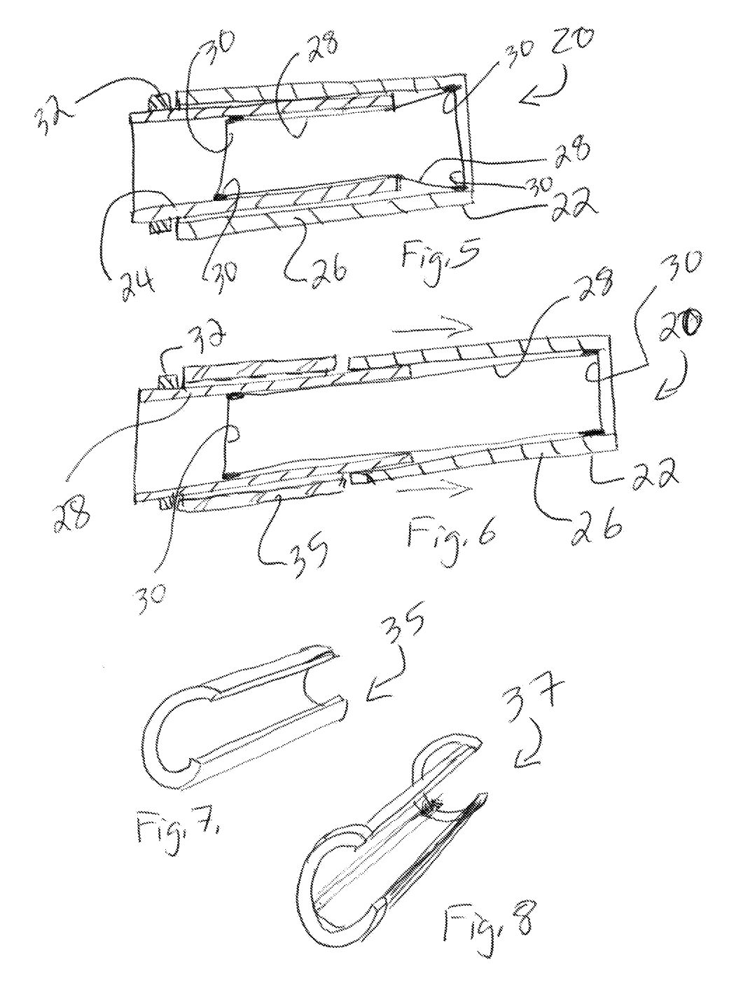

FIGS. 7 and 8 illustrate two embodiments of the optional clip or spacers 35 and 37. Either of these spacers may be used to ensure that the first and second catheter segments are maintained in an extended position.

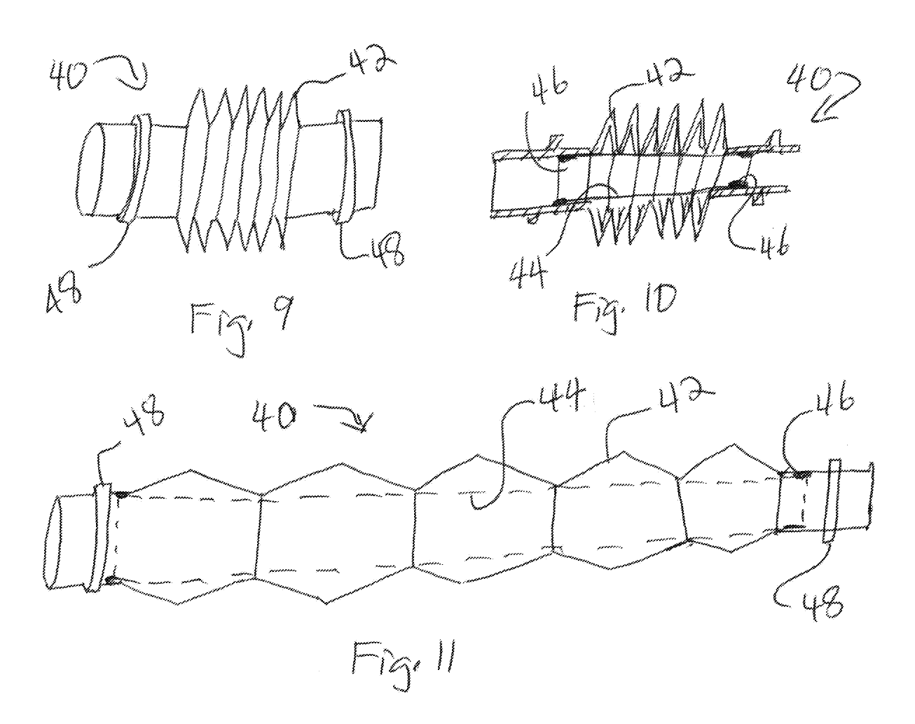

FIGS. 9-11 illustrate a corrugated (folds), adjustable length catheter portion 40 that may be integral with the catheter shaft 2 or may be separately formed and attached to the catheter shaft as a separate step. The corrugated or folded portion has corrugations or folds 42 that may be in a compressed or shortened length configuration (FIGS. 9 and 10) or an expanded or extended length configuration (FIG. 11). In both configurations, an inner liner 44 maintains a smooth surface to reduce the likelihood of thrombus formation. The liner may be of any of the materials described above.

The liner 44 is fused or otherwise attached at attachment points 46 to the inner surface of the inner lumen of the adjustable length catheter portion 40 such that in a shortened configuration (FIG. 10) the liner is generally smooth. This is desired to reduce the likelihood that a thrombus will form on the surface, as could be expected if the surface had numerous folds or was too irregular. As illustrated in FIG. 10, the liner also is smooth when the adjustable length catheter portion is in the extended or expanded position. In this configuration, the Teflon (PTFE) layer or coating of the guiding catheter is contiguous with the liner such that a continuous smooth surface is formed.

Depending upon the elasticity of the liner there may be more or less tendency in the extended configuration for the corrugations to collapse and return to a shortened configuration. To the extent such a tendency occurs and is an inconvenience to the operator, the end of the portion 40 may be provided with a ridge or multiple protrusions 48. The position of the ridge or protrusions will create a length between the opposite ridges into which a clip or spacer may be placed, thereby ensuring that the first and second catheter segments are maintained in an extended position. The clip or spacer may be one of those illustrated in FIGS. 6 and 7, above, and its use is optional.

In one embodiment of a catheter according to the invention, the catheter has the proximal adjustable length portion adjacent to the hub and only a single adjustable length portion is present. The adjustable length portion can be provided in a compressed length providing maximal extension, an extended length providing maximal length shortening, or in a partially extended configuration thereby providing the ability to both extend the length and shorten the length using a single adjustable length portion. These three configurations can be used in the embodiments described herein.

In another embodiment of a catheter according to the invention, the catheter has more than one proximal adjustable length portion adjacent to the hub such that multiple adjustable length portions are available to the operator. If two adjustable length portions are provided in the compressed or shortened configuration, the operator can extend both portions but will be unable to shorten the length of the catheter. If the two adjustable length portions are provided in the extended configuration, the operator can shorten both portions but will be unable to extend the length of the catheter. If the one of the two adjustable length portions is provided in the shortened configuration and the other is provided in the extended configuration, the operator will have the ability to both shorten and extend the length of the catheter.

It also should be understood that in one embodiment a corrugated adjustable length portion and a telescoping adjustable length portion may be used on the same catheter to provide two different way to adjust the length of the catheter.

In another embodiment the catheter is provided such that the catheter has the adjustable length portions or zones provided adjacent to the hub, thereby being proximally positioned for the operator to manipulate during a procedure. The distal end of the adjustable length portion may be within 20 cm of the hub, within 15 cm of the hub, within 14 cm of the hub, within 13 cm of the hub, within 12 cm of the hub, within 11 cm of the hub, within 10 cm of the hub, within 9 cm of the hub, within 8 cm of the hub, within 7 cm of the hub, within 6 cm of the hub, within 5 cm of the hub, within 4 cm of the hub, within 3 cm of the hub, within 2 cm of the hub or within 1 cm of the hub or less. The distal end may be measured from the first fold or corrugation, the edge of the inner or outer telescoping segments, or from a separate portion that is fused to a catheter shaft. The hub may be attached to such a separate portion.

In another embodiment the catheter is free of an adjustable portion that is distal of a position that is 20 cm from the hub or that is adjacent to the distal end of the catheter. In other words, in the alternative embodiment, the adjustable portion must be adjacent to the hub and unlikely to be within the vasculature.

The adjustable length portions or zones may be of a variety of lengths when extended, such as about 15 cm, about 14 cm, about 13 cm, about 12 cm, about 11 cm, about 10 cm, about 9 cm, about 8 cm, about 7 cm, about 6 cm, about 5 cm, about 4 cm, about 3 cm, about 2 cm or about 1 cm. Similarly, the adjustable length portions or zones may be fully extended or partially extended.

In use, the physician places an introduced within the vasculature, such as the femoral artery, and inserts a guide wire through the introducer until the guide wire reaches the target point in the vascular system. The operator then inserts the guiding catheter over the guide wire into the introducer and to the target point. If the operator determines that the catheter is too short, the operator may extend, expand or otherwise lengthen the proximal, adjustable length catheter portion to achieve the desired length. If the operator determines that the catheter is too long, the operator may compress or otherwise shorten the length of the proximal, adjustable length catheter portion to achieve the desired length. In either case, the operator can continue the procedure without need to remove the guiding catheter because of a mismatch in length.

While several particular forms of the invention have been illustrated and described, it will be apparent that various modifications and combinations of the invention detailed in the text and drawings can be made without departing from the spirit and scope of the invention. For example, references to materials of construction, methods of construction, specific dimensions, shapes, utilities or applications are also not intended to be limiting in any manner and other materials and dimensions could be substituted and remain within the spirit and scope of the invention. Accordingly, it is not intended that the invention be limited, except as by the appended claims.

I claim:

1. A hollow catheter having an inner lumen with an inner surface and comprising:
a distal catheter shaft;
an adjustable length catheter portion; and
a proximal hub,
wherein the adjustable length catheter portion is positioned between the distal catheter shaft and the proximal hub and includes a segment that is either expandable to extend the length of the catheter or is compressible to shorten the length of the catheter,
wherein the adjustable length catheter portion has a proximal end within about 10 cm of the hub, and
wherein the adjustable length catheter portion has a continuously smooth inner surface when in the expanded or shortened configuration, the smooth inner surface being formed of a biocompatible material that is stretched when the adjustable length catheter portion is expanded and is relaxed when the adjustable length catheter portion is shortened.

2. The hollow catheter of claim 1, wherein the adjustable length catheter portion has the proximal end within about 7 cm of the hub.

3. The hollow catheter of claim 1, wherein the adjustable length catheter portion has the proximal end within about 5 cm of the hub.

4. The hollow catheter of claim 1, wherein the adjustable length catheter portion has the proximal end within about 3 cm of the hub.

5. The hollow catheter of claim 1, wherein the adjustable length catheter portion has the proximal end within about 1 cm of the hub.

6. The hollow catheter of claim 1, wherein the biocompatible material forming the smooth inner surface of the adjustable length catheter portion is a urethane.

7. The hollow catheter of claim 1, wherein the biocompatible material forming the smooth inner surface of the adjustable length catheter portion is a polyether block amide.

8. The hollow catheter of claim 1, wherein the biocompatible material forming the smooth inner surface of the adjustable length catheter portion is a rubber.

9. The hollow catheter of claim 1, wherein the adjustable length catheter portion comprises an outer catheter wall and the smooth inner surface adjacent to and coplanar with the outer catheter wall, and the outer catheter wall comprises one or more corrugations.

10. The hollow catheter of claim 1, wherein the adjustable length catheter portion comprises a first catheter segment having a first outer diameter and a second catheter segment having an inner diameter that is greater than the first outer diameter such that the second catheter segment can telescope over the first catheter segment.

11. The hollow catheter of claim 10, wherein the smooth inner surface is attached to the first catheter segment and the second catheter segment such that a continuous and smooth inner liner is formed when the two catheter segments are expanded and shortened.

12. The hollow catheter of claim 1, wherein the adjustable length catheter portion comprises an adjustable zone formed by a plurality of circumferential corrugations in accordion-like ridges and grooves whereby the catheter may be lengthened by increasing the distance between the ridges or shortened by decreasing the distance between the ridges.

13. The hollow catheter of claim 12, wherein each ridge is comprised of a long side and a short side such that each ridge stacks neatly into an adjacent ridge, whereby the stacked ridges provide the operator with an an easy grasp on the adjustable zone and the ability to change or maintain the selected catheter length in increments.

14. A method of adjusting a length of a catheter while in a patient, the method comprising:
providing a catheter having an inner lumen with an inner surface and including a distal catheter shaft, an adjustable length catheter portion, and a proximal hub, wherein the adjustable length catheter portion is positioned between the distal catheter shaft and the proximal hub and includes a segment that is either expandable to extend the length of the catheter or is compressible to shorten the length of the catheter, wherein the adjustable length catheter portion has a proximal end within about 10 cm of the hub, and wherein the adjustable length catheter portion has a continuously smooth inner surface when in the expanded or shortened configuration, the smooth inner surface being formed of a biocompatible material that is stretched when the adjustable length catheter portion is expanded and is relaxed when the adjustable length catheter portion is shortened;
inserting the catheter into a tubular vessel of a patient; and
adjusting the length of the catheter without removing the catheter from the vessel by adjusting the adjustable length catheter portion.

15. The method of claim 14, wherein the adjustable length catheter portion has the proximal end within about 7 cm of the hub.

16. The method of claim 14, wherein the adjustable length catheter portion has the proximal end within about 5 cm of the hub.

17. The method of claim 14, wherein the adjustable length catheter portion has the proximal end within about 3 cm of the hub.

18. The method of claim 14, wherein the adjustable length catheter portion has the proximal end within about 1 cm of the hub.
